(19) United States
(12) Patent Application Publication
(10) Pub. No.: US 2011/0230730 A1
(43) Pub. Date: Sep. 22, 2011

(54) Method, System and Computer Program Product for Limb Movement Analysis for Diagnosis of Convulsions

(75) Inventors: Mark S. Quigg, Charlottesville, VA (US); Michael L. Johnson, Keswick, VA (US); Daniel Redmond, Silver Spring, MD (US); William W. Campbell, North Bethesda, MD (US)

(73) Assignee: UNIVERSITY OF VIRGINIA PATENT FOUNDATION, Charlottesville, VA (US)

(21) Appl. No.: 12/671,788

(22) PCT Filed: Aug. 1, 2008
(86) PCT No.: PCT/US08/72004

§ 371 Date: Apr. 5, 2010

Related U.S. Application Data
(60) Provisional application No. 60/963,284, filed on Aug. 3, 2007.

(51) Int. Cl. A61B 5/00

(52) U.S. Cl. 600/301

(57) ABSTRACT

A method or system is provided for diagnosing convulsions of a subject. The method or system may include two general components. First, a device or apparatus that measures and accumulates an electronic data stream representing the physical movements of the human limb(s) and presents that data to a suitable computational device or system for further processing. Second, a set of software or firmware algorithms that operates within the computational device on the data stream for the purpose of describing and characterizing the statistical properties of the movement signal during clinical seizure episodes. The system comprised of both components serves to distinguish and diagnose the movement features of ES events from those of PNES.

### RELATED APPLICATIONS

The present invention claims priority from U.S. Provisional Application Ser. No. 60/963,284, filed Aug. 3, 2007, entitled “Method and System for Limb Movement Analysis for Diagnosis of Convulsions;” the disclosure of which is hereby incorporated by reference herein in its entirety.

### BACKGROUND OF THE INVENTION

Almost ten percent of the U.S. population will experience at least one seizure during their lifetime. While seizures can manifest in many ways, the abrupt onset of altered or lost consciousness along with motor activity—a convulsion—is a common clinical manifestation of a seizure.

Accurate diagnosis remains a problem. Epileptic seizures (ES) arise from abnormal, hypersynchronous bursts of brain activity, and, once diagnosed, usually are treated with anticonvulsant medications. However, a number of medical conditions can give rise to events that can mimic ES. Psychogenic nonepileptic pseudoseizures (PNES) are associated with psychiatric disorders. In contrast to ES, brain activity remains normal during a PNES, and anticonvulsant medications are not effective in treatment. Clinical observation of convulsive movements may be helpful but are not reliable in diagnosis.

Electroencephalography (EEG) is the main tool used by neurologists to support diagnoses of ES. Most findings on routine EEG pertinent to epilepsy are interictal (between seizures). The sensitivity of a single, routine EEG in known epilepsy is about 60%. The sensitivity may increase to a maximum of about 80% after repeat recordings. Ictal EEG recordings (those capturing seizures) may be diagnostic, since clinical and electrographic activities may be directly correlated. The problem, of course, is that the likelihood of an epileptic seizure occurring within the limited duration of a routine recording is low. To maximize the possibility of capturing a seizure event, continuous video-EEG (CV-EEG) is the gold standard in the differential diagnosis of seizures. In the UVA epilepsy monitoring unit, over one-third of our admissions are dedicated to this problematic patient subgroup.

Distinct limitations, however, prevent the successful use of CV-EEG. First, patients require several days of monitoring while admitted to the hospital, an inconvenient and expensive process. Second, patients with rare events may not qualify for CV-EEG since the probability of capturing events in a practical period of time is rare. Third, patients who have major motor activity during events may make interpretation of CV-EEG data difficult because of movement and muscle artifacts. Fourth, CV-EEG requires the application of scalp electrodes; the intense maintenance of electrode quality and the possibility of skin reactions to long term electrode application limits long term, unsupervised use of CV-EEG.

Therefore, a means of monitoring patients with convulsions of unknown origin that allows remote, noninvasive, and long term data collection may aid in diagnosis of these patients.

### BRIEF SUMMARY OF INVENTION

An aspect of an embodiment of the present invention provides limb accelerometry combined with movement analysis may offer an important adjunctive procedure in this patient group. Our current research, using a particular embodiment of the present method and system, demonstrates the quantification of differences in motor activity which distinguish ES from PNES.

The present method and system invention is comprised of two general components: First is a device or apparatus that measures and accumulates an electronic data stream representing the physical movements of the human limb(s) and presents that data to a suitable computational device or system for further processing; and Second, a set of software or firmware algorithms that operates within the computational device on the data stream for the purpose of describing and characterizing the statistical properties of the movement signal during clinical seizure episodes. The system comprised of both components serves to distinguish and diagnose the movement features of ES events from those of PNES.

An embodiment of the present invention system and method may comprise, but is not limited thereto, the following components: 1) Device: a wrist-mounted accelerometer and 2) Software: algorithms that quantify the regularity and rhythmicity of motor activity.

Various embodiments of the present invention system and method provide for, but not limited thereto, a novel application of devices (e.g., accelerometer) with a variety of software algorithms. Diagnosis between ES and PNES is possible by the characterization of patient movements, with ES having more irregular and lower frequency movements.

An aspect of the present invention is that it is the first system and method that may offer clinically-useful sensitivity and specificity for diagnosis of ES vs. PNES outside of the inpatient monitoring unit, and thus may be embodied in devices and systems that are fully portable, non-encumbering to the patient, and relatively inexpensive.

An aspect of an embodiment of the present invention provides a computer implemented method of distinguishing epileptic (ES) from non-epileptic pseudoseizures (PS) derived from motor activity of the limbs of a subject. The method may comprise measuring and accumulating an electronic data stream representing said motor activity. The method may further comprise quantifying differences within said electronic data stream to distinguish said epileptic (ES) from non-epileptic pseudoseizures (PS). The measuring may be provided by a sensor device. The sensor device may be at least one of the following: accelerometer, electromyographic electrodes or optoelectronic distortion sensors. The sensor device may be place on a limb of said subject as desired or required.

An aspect of an embodiment of the present invention provides a system for diagnosing convulsions of a subject. The system may include a sensor device for measuring and accumulating an electronic data stream representing motor activity; and a processor for processing said electronic data to distinguish epileptic (ES) from non-epileptic pseudoseizures (PS). The sensor device may be at least one of the following types of devices: accelerometer, electromyographic electrodes or optoelectronic distortion sensors. The processing may include an approximate entropy (ApEn) technique or a peak-to-peak amplitude of repeated patterns (PPARP) technique, or any combination thereof.

An aspect of an embodiment of the present invention provides a computer program product comprising a computer useable medium having computer program logic for enabling at least one processor in a computer system to diagnose convulsions of a subject. The computer program logic may include: receiving electronic data representing motor activity; and processing said electronic data to distinguish epileptic (ES) from non-epileptic pseudoseizures (PS). The processing may be accomplished using an approximate entropy (ApEn) technique or peak-to-peak amplitude of repeated patterns (PPARP) technique, or any combination thereof.

These and other objects, along with advantages and features of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1(A) schematically represents the steps or modules for carrying out an embodiment of the present invention Convulsion Diagnosis.

FIG. 1(B) schematically represents the steps or modules for carrying out an embodiment of the present invention Convulsion Diagnosis.

FIG. 2 graphically illustrates ApEn values during the total course of spells of ES (solid line/dash-dot-dash line) vs. nonepileptic (dash line) from a pilot study.

FIG. 3 graphically illustrates the time-domain measure of signal complexity in subgroup of 19 patients from the pilot study.

FIG. 4 provides a schematic diagram illustrating a system in which examples of the invention can be implemented.

FIG. 5 provides is a diagram showing an exemplary computing device having computer-readable instructions in which example of the invention can be implemented.

### DETAILED DESCRIPTION OF THE INVENTION

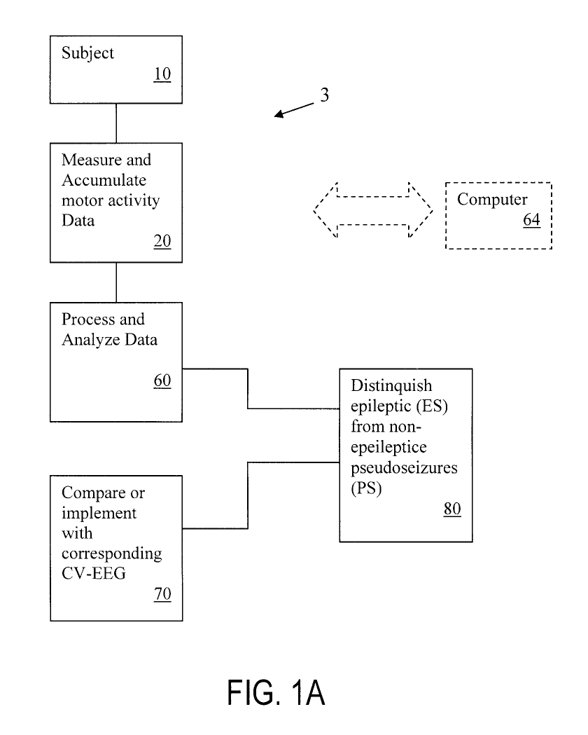

In general, referring to FIG. 1(A), an aspect of an embodiment of the diagnostic convulsion system and related method that provides for measuring and accumulating an electronic data stream 20 representing and motor activity of subject 10; and processing and analyzing 60 said motor activity electronic data to distinguish epileptic (ES) from non-epileptic pseudoseizures (PS) 80. The distinguished data may be compared to CV-EEG 70 and/or implemented with subsystems of the CV-EEG 70.

### Movement Detection Device or Apparatus

Referring to FIG. 1(B), a sensor module 22 may be a device or apparatus that measures physical movement of a subject 10 by providing electronic data representing the movement. The sensor module and/or its related components (along with an integrated processor or separate processor) accumulate the electronic data stream representing the physical movements. For instance, the sensor module 22 (and related processing) may be a device or apparatus that measures and accumulates an electronic data stream representing the physical movements of the human limb(s) is the first stage of an exemplary embodiment this invention. Such a component in this apparatus may be a sensor which converts physical movements into an electrical voltage signal which instantaneously varies in relation to the amplitude and frequency of the movement. One class of sensors, termed accelerometers, produces a signal proportional to changes in the velocity of the sensor. Other classes of sensors, such as electromyographic electrodes or optoelectronic distortion sensors, may serve equally well to produce the required electronic data stream. It should appreciated that a variety of available sensors may be implemented as desired or required and would be considered within the context of the present invention.

In the embodiment used in the previous example (prototype), the sensor was comprised of a commercial piezoresistive accelerometer (Model ICS 3150-002, Measurement Specialties, Inc., Fairfield, N.J.). Other sensors of this class may be used to generate signals similarly in embodiments of this invention. For instance, a bi-morph piezoelectric beam has been used to measure and record body movements (see Conlan, U.S. Pat. No. 5,197,489, of which is hereby incorporated by reference herein in its entirety). Also, a micro-miniature sensor device, producing individual accelerometric signals for all three directions of movement (x, y, and z axes), is available for self-contained, portable embodiments (e.g., Model ADXL330, Analog Devices, Inc., Norwood, Mass.).

A conditioning processor module 32 is provided to receive data stream from the sensor module 22 (and associated processing) or desired sensing or measuring means for the purpose of describing and characterizing the statistical properties of the movement signal during clinical seizure episodes of the subject. For example, but not limited thereto a processor module 32 may be a Conditioning Electronic Circuitry (CC) to which the sensor 22 is coupled (or in communication therewith of some type of hardware or wireless), which conditions the signal such that it is suitable for the computations and analyses that follow. Characteristics of the pre-conditioned signal, determined by the design of this circuitry of the processor module 32, include the following:
1. Sensitivity, Voltage Gain, and Voltage Range. Sensitivity is an intrinsic property of the sensor specified defining the quantitative linear relationship between movement and sensor output signal, expressed (for accelerometers) as millivolts per g. Voltage Gain is achieved by amplifier(s) coupled to the sensor, and is designed to 1) achieve a Voltage Range (VR) (volts per g) equivalent to the maximum expected range of movement acceleration (e.g., +/−3 g); 2) a VR that is matched to the Input Voltage Range of the Digitization Circuitry (v.i.); and 3) is linear over the VR, and avoids any excursions beyond the Digitization Input Range which would distort the subsequent computations and analyses.
2. Frequency Pass Band. Bodily movement is limited in frequency, even in cases of very rapid tremor and shivering, by the biophysics of muscular action, to a value below about 20 cycles per second. (See J. Timmer et al., “Quantitative Analysis of Tremor Time Series,” Electroencephalography and Clinical Neurophysiology, 101, Mar. 20, 1996, pp. 461-468 (of which are hereby incorporated by reference herein in their entirety)—and related references). Thus, the CC provides attenuation of the frequency content of the signal to values above about 20 Hz by low-pass filtering. The exemplary embodiment uses a simple resistor-capacitor (RC) filter with a cutoff of 36 Hz. In addition, transient changes in the static position of the sensor (or posture) with respect to gravity produce deflections in sensor output equivalent to a range of +/−1 g. Thus, the CC provides attenuation of frequency below a normal range of dynamic movements, of about 0.5 or 1.0 cycles per second. The exemplary embodiment achieves this by coupling the accelerometer output signal to an AC channel as part of the overall EEG montage. An embodiment suitable for application of this invention to analysis of Epileptic Seizure Events should provide a passband of 1.0 to 16 Hz.

A feature of the combined sensor module 22 and signal conditioning processor module is the ability to adjust and calibrate the output signal with an output of constant amplitude, for consistency within the same apparatus across time and usage, and for uniformity across different instances or versions of the apparatus.

Another component of this apparatus is a converter module 42 that converts the signals from the conditioning processor module 32 into a discrete numbers. For instance, the converter module 42 may be an electronic digitization circuitry which contains an analog-to-digital converter that converts the conditioned sensor signal, a varying analog voltage, into a data stream or series of discrete numbers. This may be accomplished by sampling the instantaneous voltage at a frequency at least twice that of the CC low-pass frequency. The exemplary embodiment uses 200 samples per second, while a suitable embodiment would be at a minimum of 32 samples per second, or a magnitude as desired or required. Each sample is electronically converted into a binary number, scaled such that the Voltage Range is divided by a resolution value of 2ⁿ, where n is the number of binary bits associated. Thus the exemplary embodiment utilizes a 12-bit converter, and divides the Input Voltage Range into 4096 discrete values, so that each sample in the data stream lies numerically between 0 and 4095.

The physical location of the converter module 42, e.g., digitization circuitry, within this apparatus may be one of two places:
1. In the exemplary embodiment, digitization occurs in the commercial CV-EEG recording system. Thus, the Sensor 22 and Conditioning 32 Circuitry, with its analog voltage signal, is tethered to the system over an electrical cable, coupled to the input amplifier box. Final conditioning 32 and digitization 42 occurs in this input stage, and then the CV-EEG proceeds to record the data stream into the Recording Subsystem 52.
2. In a self-contained, portable embodiment, the Digitization Circuitry 42 is co-located with and is a physical extension of the Conditioning Circuitry 32, as is the Recording Subsystem 52.

Still referring to FIG. 1(B), a recording module 52 may be another component of the system. Such a recording module 52 may be a recording subsystem that may include a means of accumulating and recording the numeric data stream output by the converter module 42 (e.g., digitization circuitry) so that it is retrievable following a recording session, and can be presented to a computational system (e.g., PC) that can execute the processing and analysis algorithms of this invention.

In the exemplary embodiment, the CV-EEG records the data directly into data files which are written to intrinsic magnetic and/or optical recording media, under the operational control of software programs intrinsic to the commercial CV-EEG system. These files are subsequently downloaded into the Analysis System 62.

In a self-contained, portable embodiment, electronic digital memory chips may be used to record the data stream, and comprise a local Recording Subsystem 52, under the operational control of a microcontroller chip and its associated firmware program. Data contained in memory are subsequently downloaded into the Analysis System 62 over an appropriate communications interface, by wire, or by radio- or optical-telemetry and converted to data files.

A feature of the combined Digitization component 42 and Recording component 52 is the ability to mark the digital data stream with time coordinates, providing both clock time of data sample acquisitions, and the sampling frequency of digitization. This information is contained in the associated data files for each recording session.

A feature of the combined Digitization component 42 and Recording component 52 is the ability to mark the digital data stream with Event Marks, which define the temporal boundaries of seizure or pseudo-seizure events. These marks are entered manually by either the patient or a reliable observer. This information is contained in the associated data files for each recording session.

While the exemplary embodiment involves only one axis and device location, namely movement along the flexor-extensor axis of the wrist, another embodiment includes movements in two orthogonal axes. In the case of wrist emplacement, this would add sensitivity to movement along the radio-ulnar axis. This implies the use of two “channels” of sensor, signal conditioning, digitization, and recording functions.

### Clinic or Environment Set-up

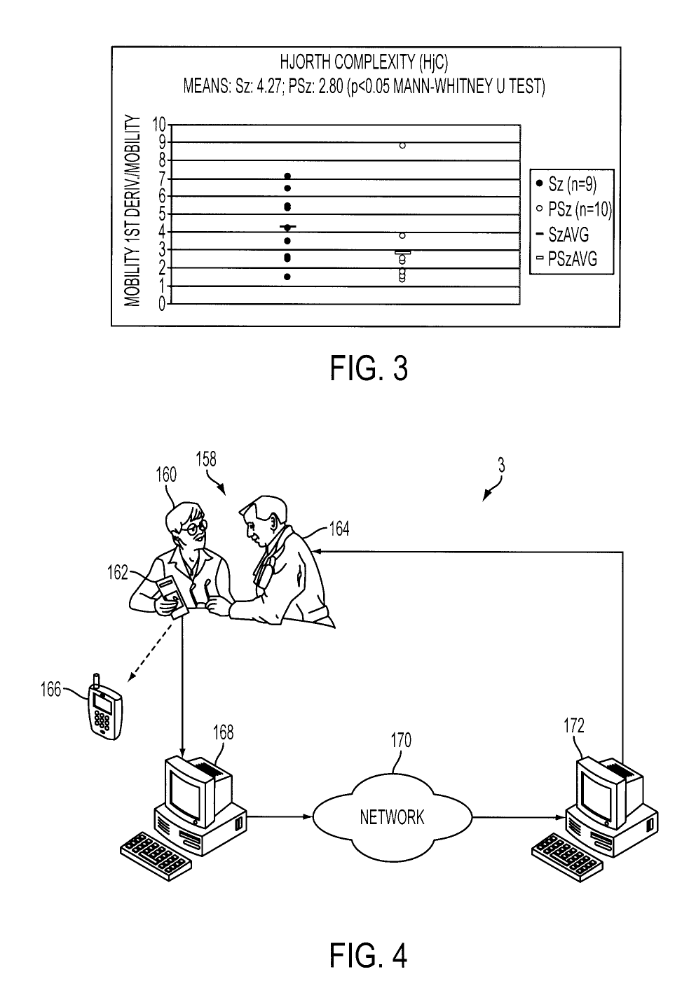

FIG. 4 diagrammatically illustrates an exemplary system in which examples of the invention can be implemented. Referring to FIG. 4, clinic setup 158 provides a place for doctors (e.g. 164) or clinicians to diagnose subjects or patients (e.g. 160) undergoing vEEG testing or selected for other reasons to measure seizure movements.

A sensor 162 (or accelerometer or other device) may be worn by or placed on a subject 160 that can be used to measure limb movements of the subject (or other areas of the subject as desired or required) for the purposes of diagnosing or analyzing convulsions. Such sensors and/or test can be short term (e.g. clinical visit) or long term (e.g. clinical stay or family). The sensor 162 (and/or other portions of the diagnostic convulsion system 3) incorporates the improvement so as to demonstrate the quantification differences in motor activity which distinguish ES from PNES. The sensor 162 (and/or other portions of the diagnostic convulsion system 3) outputs with improved accuracy of convulsion diagnosis and analysis that can be used by the doctor (or other clinicians) for appropriate actions for treatment or diagnosis of the subject or patient 160. It should be appreciated that the sensor 162 may be worn like a watch or attached to other limbs (or other areas) as desired or required.

Alternatively, the sensor 162 output (and/or output of other portions of the diagnostic convulsion system 3) provides improved accuracy and information that can be delivered to computer terminal 168 for instant or future analyses, diagnosis and treatment. The delivery can be through cable or wireless or any other suitable medium. The sensor 162 output (and/or output of other portions of the diagnostic convulsion system 3) with its improved accuracy for diagnosis, analyses, and treatment concerning the patient can also be delivered to a portable device, such as PDA 166. The sensor 162 output (and/or output of other portions of the diagnostic convulsion system 3) can similarly be delivered to a monitoring or diagnostic center 172 for processing and/or analyzing, or other desired or required applications. Such delivery can be made accomplished through many ways, such as network connection 170, which can be wired or wireless.

In addition to the sensor 162 output (and/or output of the other portions of the diagnostic convulsion system 3) and related information can be delivered, such as to computer 168, and/or data processing center 172 for diagnosis convulsions. This can provide a centralized accuracy monitoring and/or accuracy enhancement for diagnostic and treatment centers, or other activity as desired or required.

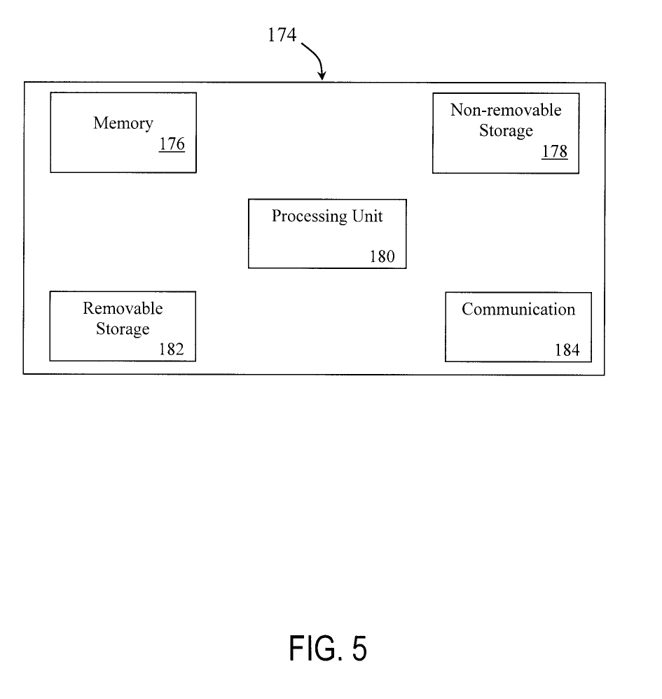

As discussed earlier, examples of the invention can also be implemented in a standalone computing device associated with the target motion sensors 162 or accelerometers. An exemplary computing device in which examples of the invention can be implemented is schematically illustrated in FIG. 5. Although such computing devices 174 are generally well known to those of skill in the art, a brief explanation will be provided herein for the convenience of other readers.

Referring to FIG. 5, in its most basic configuration, computing device 174 typically includes at least one processing unit 180 and memory 176. Depending on the exact configuration and type of computing device, memory 176 can be volatile (such as RAM), non-volatile (such as ROM, flash memory, etc.) or some combination of the two.

Additionally, device 174 may also have other features and/or functionality. For example, the device could also include additional removable and/or non-removable storage including, but not limited to, magnetic or optical disks or tape, as well as writable electrical storage media. Such additional storage is the figure by removable storage 182 and non-removable storage 178. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology, CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of or used in conjunction with, the device.

The device may also contain one or more communications connections 184 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. As discussed above, the term computer readable media as used herein includes both storage media and communication media.

In this document, the terms “computer program medium” and “computer usable medium” are used to generally refer to media or medium such as various software, firmware, disks, drives, removable storage drive, a hard disk installed in hard disk drive, and signals. These computer program products (“computer program medium” and “computer usable medium”) are means for providing software to computer system. The computer program product may comprise a computer useable medium having computer program logic thereon. The invention includes such computer program products. The “computer program product” and “computer useable medium” may be any computer readable medium having computer logic thereon.

### Software Algorithms and Methodologies for Processing and Analysis of Data

Processing and Analysis are performed by computational system module 64, such as a PC for example, that may utilize PC software using standard commercial and customized software programs. It should be appreciated that a variety of types of processors may be employed within the context of the invention.

Data retrieved from the data files consist, for each “channel,” of a time series of numerical values, with a number of data points equal to the length of the recording session, in seconds, times the sampling rate, in samples per second. These large data series are subdivided in two ways: 1) by breaking data into “windows” of m contiguous values, sliding the window across the data and performing Processing and Analysis on the subsets of data contained in each successive window; and 2) by defining a unique window for each ES or PNES event, bound by Event Markers or clinical annotations, and performing Processing and Analysis by the computational system module 64 on the ensemble of data contained within each Event Window.

Digital preprocessing of data may be performed on subsets of data appropriate to the subsequent analytical algorithms to be applied. These processes include normalization of data sets and Fourier transforms

Diagnostic clinical inferences, with regard to ES and PNES discrimination, will increase in specificity and selectivity as the clinical data base increases in scope through ensuing research.

### Analytical Algorithms:

1) Two exemplary and non-limiting algorithms are applied to these data which are specifically suited to the diagnostic purpose, based on sensitivity to variation of regularity/complexity of movements in such data:
   a) Approximate entropy (ApEn) measures the consistency of pattern recurrence within a tolerance range r for m contiguous values (run length); ApEn values were normalized by dividing the absolute ApEn value by the mean ApEn from 1000 randomly shuffled surrogates of the same data series. Normalized ApEn rations of unity approach maximum randomness, whereas values closer to zero denote more orderly sequences.
   b) The peak-to-peak amplitude of repeated patterns (PPARP) technique iteratively fitted peak-peak amplitude of a sliding window of m contiguous values divided by the residual fractional variance of the fitted amplitude. In this scheme, high values result from large amplitude component of activity in the nominator or from a small variance of whatever in the denominator, consistent with a simple, high-amplitude sinusoidal rhythm. Low values describe small amplitude activities with large variances in fit, consistent with complex or disordered activity.
2) More generic analyses based on the statistical characteristics of data sets, often applied to EEG and EMG signals, are included in the analytical approach to the data:

Provided below are computational methods used for the exemplary embodiments of the present invention:

### Computational Methods

### A. Time Domain Measures

1. Root Mean Squared Voltage (RMSV) is the standard deviation of the voltage signal V(t) for all regular samples t collected during signal acquisition:

$$R M S V = {[ \frac{{\sum }_{t = 1}^{N} {[ V ( t ) - \overline{V ( t )} ]}^{2}}{N} ]}^{1 / 2} .$$

2. Mean Rectified Voltage[a] (MRV) is the mean amplitude of the rectified signal:

$$M R V = \frac{1}{N} {\sum }_{t = 1}^{N} \vert V ( t ) \vert .$$

3. Coefficient of Variation (CVAR) of the signal is calculated from the standard deviation of the rectified signal:

$$C V A R = \frac{{[ \frac{{\sum }_{t = 1}^{N} {[ \vert V ( t ) \vert - M R V ]}^{2}}{N} ]}^{1 / 2}}{M R V} .$$

4. Coefficient of Sequential Variation[b] (CSV) is similar, but calculated from sequential pairs of samples, by calculating the Root Mean Square of Successive Differences (RMSSD):

$$R M S S D = {[ \frac{{\sum }_{t = 1}^{N - 1} {[ V ( t + 1 ) - V ( t ) ]}^{2}}{N - 1} ]}^{1 / 2}$$

$$Then , C S V = \frac{R M S S D}{M R V} .$$

5. Zero Crossing Frequency[a] (ZC) is obtained by counting the number of times the signal changes sign, or transitions across zero from either direction, =N_{Z}, then is expressed as a count per second:

$$ZC = \frac{{N}_{Z} \cdot SamplingRate}{2 N} {seconds}^{- 1} .$$

6. Turns[a] are obtained identically, using the First Derivative of the Signal.

7. Hjorth[c] Activity is the amplitude variance of the signal, equivalent to RMS voltage.

8. Hjorth[c] Mobility is the square root of the variance of the 1st Derivative, divided by the Activity.

9. Hjorth[c] Complexity (or Form Factor) is the Mobility of the 1st Derivative, divided by the Mobility.

### B. Frequency Domain Measures

Descriptors of the Power Spectrum. The AutoPower Spectrum was computed using a Visual Studio Measurement Studio function to transform the original time-series signal data to the power-frequency domain. The resulting transform, P(f), allows a description of the original signal in terms of frequency content and complexity that may discriminate between the ES and PS groups.

1. Normalized Power[a] (nPWR) is the mean power calculated from the Power Spectrum P(f):

$$nPWR = \frac{1}{N} [ \frac{1}{{f}_{H} - {f}_{L}} ] {\sum }_{f = fL}^{fH} P ( f ) .$$

2. Median Frequency[a] (MF) is the frequency value that divides the spectrum (P(f)) in two halves of equal areas, so that:

$${\sum }_{f = fL}^{MF} P ( f ) = {\sum }_{f = MF}^{fH} P ( f ) .$$

3. Mean Power Frequency[a] (MPF) is the Average of Frequencies, weighted by Power (f):

$$M P F = [ \frac{{\sum }_{f = fL}^{fH} f \cdot . P ( f )}{{\sum }_{f = fL}^{fH} P ( f )} ] .$$

4. Expected Zero Crossing Frequency[a] is the 2^{nd} Moment of spectral components:

$$E Z C = {[ \frac{{\sum }_{f = fL}^{fH} {f}^{{2}_{2}} \cdot P ( f )}{{\sum }_{f = fL}^{fH} P ( f )} ]}^{1 / 2}$$

5. Complexity[a] is the 4^{th} Moment of spectral components:

$$Complexity = {[ \frac{{\sum }_{f = fL}^{fH} {f}^{4} \cdot P ( f )}{{\sum }_{f = fL}^{fH} P ( f )} ]}^{1 / 2}$$

6. Power Variance[a] (PVAR) the average deviation of P(f) from the mean, squared:

$$P V A R = \frac{{\sum }_{f = fL}^{fH} {[ P ( f ) - \overline{P ( f )} ]}^{2}}{{N}_{f}} ,$$

Where N_{f} is the number of frequencies between f_{L} and f_{H}.

7. Coefficient of Power Variance[a] (cPVAR) is PVAR divided by the Amplitude Variance:

$$c P V A R = \frac{{[ P V A R ]}^{1 / 2}}{R M S}$$

Time-Frequency Mapping[d]. Vinton et al. (2004)¹, using EEG movement artifact signals, proposed the segmentation of the data into discrete epochs of a few seconds, and for each epoch, the power spectrum was derived, and the frequency with peak power was recorded. For the whole episode, the mean and standard deviation of these peak frequencies, and their coefficient of variation, were calculated. For the present study with accelerometer data, we chose an epoch of 3 seconds. The resulting measures reflect the over-all frequency of movement, its stability, or conversely, its complexity over time:
1. Mean Peak Frequency (PKF) is the average of frequencies with maximum power, obtained from serial 3-second epochs within the seizure episode.
2. Coefficient of Variation of Peak Frequencies (CVF) is the standard deviation of PKF divided by the mean.

### EXAMPLES AND EXPERIMENTAL RESULTS

Practice of the invention will be still more fully understood from the following examples and experimental results, which are presented herein for illustration only and should not be construed as limiting the invention in any way.

### Example No. 1

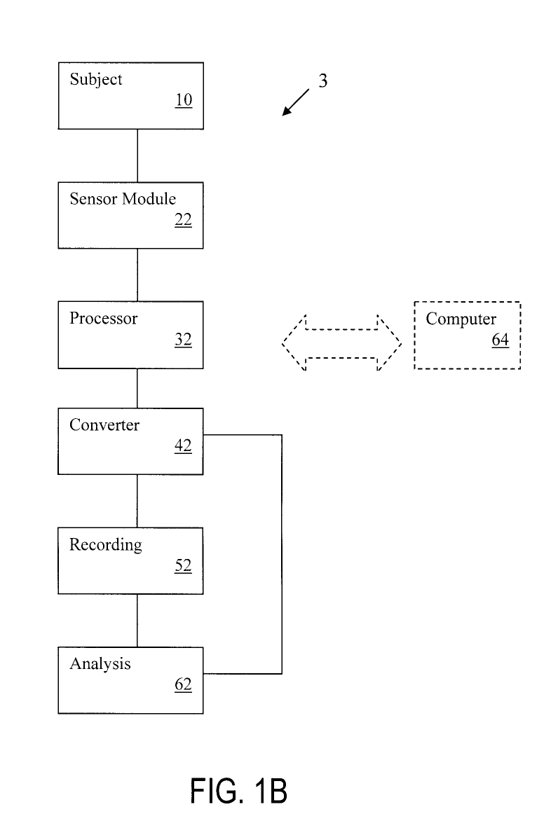

For instance, in a pilot study, patients between ages 16-60 years were admitted for CV-EEG diagnosis of convulsions. Seizure episodes selected for analysis consisted of generalized gross motor activity. A data stream representing wrist movements during and between seizures was obtained with the use of a wrist-mounted accelerometer and recorded within the CV-EEG system. The temporal profile of regularity of movements was characterized by calculating, in a computer, several statistical parameters reflecting regularity and rhythmicity, including approximate entropy (ApEn) and peak-to-peak amplitude of repeated patterns (PPARP). Referring to FIG. 2, FIG. 2 graphically illustrates ApEn values during the total course of spells of ES (solid line/dash-dot-dash line) vs. nonepileptic, PS (dash line).

From 19 PNES and 15 ES patients evaluated, ES movements were significantly more irregular than PNES (mean ApEn ES=1.78, PNES=1.38, p=0.0027; mean log PPARP ES=1.72, PNES=2.80, p=0.005 Mann-Whitney U test). A Threshold Value for the minimum ApEn during an event of 1.4 distinguished ES from PNES with Se=86% and Sp=74%. The mean log PPARP threshold was1.6 for Se=73% and Sp=84%.

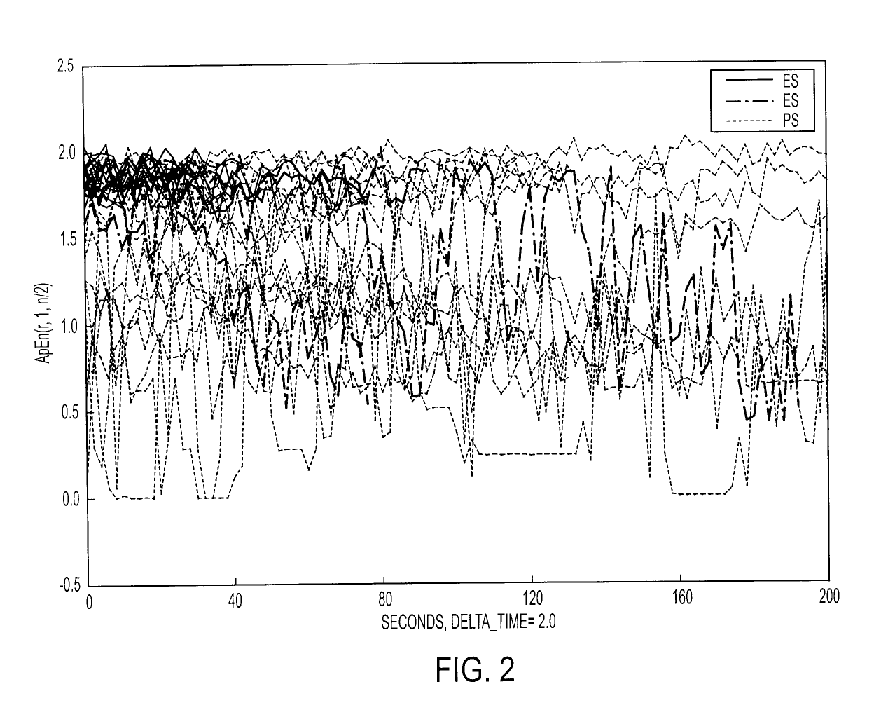

Referring to FIG. 3, for a subset of 19 of these patients, other parameters of signal regularity and complexity were calculated, and showed significant difference (p<0.05) between ES and PNES groups: Coefficient of Sequential Variation, Zero-Crossings, Hjorth Mobility, and Hjorth Complexity (all time-domain measures), and Mean Power Frequency, Expected Zero Crossings, Frequency Complexity, Mean Peak Frequency, and Coefficient of Variation of Peak Frequency (all frequency domain measures). FIG. 3 graphically illustrates the time-domain measure of signal complexity in subgroup of 19 patients.

These results are consistent with a tendency for pseudoseizure events to display movement signals having somewhat higher frequency and more regularity (or less complexity) than true epileptic seizures, a fact effectively detected by this embodiment of the present invention.

### Example No. 2

In an exemplary embodiment and study, the actigraphy system and related method are utilized in distinguishing epileptic from non-epileptic seizures. The present approach is adapted to directly measure the seizure movements, using a wrist mounted accelerometer and recording its signal along with the other vEEG channels. The present exemplary embodiment system is implemented to identify characteristics of the movement signal that might be used to devise a computational algorithm to aid in discriminative diagnosis of ES and PS. In the present study, patients were selected from those undergoing vEEG testing for clinical purposes and asked to wear a wrist mounted accelerometer added to the standard vEEG system. Resulting vEEG records were collected and reviewed by a board certified neurologist, who identified seizure events, recorded times of onset and cessation, and classified the cases as ES or PS according to standard clinical guidelines. Digitized accelerometer signal recordings were extracted from the recording, defined by each seizure event, providing data sets for further analysis.

Each event's data set was analyzed by computing a series of parameters which describe the time, amplitude and frequency characteristics of the signal. The computations, both time- and frequency-domain, included standard descriptive statistics but added parameters previously defined in the literature of signal analysis for EEG′[2]′[3], EMG [4], and EEG Artifact[1]. For each parameter, results for ES and PS groups were compared using the Mann-Whitney U test.

The results from a total of 53 events, included only 9 ES and 10 PS events were suitable in terms of duration and signal quality for further analysis. ES were complex partial seizures, with 5 having secondary generalization. The following parameters from this small sample showed significant difference (p<0.05) between groups: Coefficient of Sequential Variation, Zero-Crossings, Hjorth Mobility, and Hjorth Complexity (all time-domain measures), and Mean Power Frequency, Expected Zero Crossings, Frequency Complexity, Mean Peak Frequency, and Coefficient of Variation of Peak Frequency (all frequency domain measures).

The following patents, applications and publications are hereby incorporated by reference in their entirety herein. The devices, systems, computer program products, and methods of various embodiments of the invention disclosed herein may utilize aspects disclosed in the following U.S. patents, foreign patents, and publications and are hereby incorporated by reference herein in their entirety:

U.S. Pat. No. 6,361,508 B1 to Johnson, et. al., entitled “Personal Event Monitor With Linear Omnidirectional Response,” Mar. 26, 2002.

U.S. Pat. No. 5,197,489 to Conlan, entitled Activity monitoring apparatus with configurable filters, Mar. 30, 1993

U.S. Pat. No. 5,573,013 to Conlan, entitled Method of monitoring body movements using activity monitoring apparatus

U.S. Pat. No. 6,293,150 to Conlan, entitled Motion sensor and method of making same

U.S. Pat. No. 6,561,992 to Eberhart, et al., entitled Method and apparatus utilizing computational intelligence to diagnose neurological disorders

The following patents, applications, references and publications as cited throughout this document are hereby incorporated by reference in their entirety herein. The devices, systems, computer program products, and methods of various embodiments of the invention disclosed herein may utilize aspects disclosed in the following U.S. patents, foreign patents, and publications and are hereby incorporated by reference herein in their entirety:
(a) Baharav A, Shinar Z, and Akselrod S. Muscle Activity During Sleep: A Quantitative Electromyography Analysis during Standard Polysomnography. SLEEP 2003; 26 (Absts):A393.
(b) Luczak H and Lauring W. An Analysis of Heart Rate Variability. Ergonomics 1973; 16:85-97.
(c) Hjorth B. EEG analysis based on time domain properties. Electroencephalography and Clinical Neurophysiology 1970; 29:306-310.
(d) Vinton A, Carino J, Vogrin S, MacGregor L, Kilpatrick C, Matkovic Z, and O'Brien T. “Convulsive” Nonepileptic Seizures Have a Characteristic Pattern of Rhythmic Artifact Distinguishing Them from Convulsive Epileptic Seizures. Epilepsia 2004; 45(11):1344-1350.
1. Vinton A, Carino J, Vogrin S, MacGregor L, Kilpatrick C, Matkovic Z, and O'Brien T. “Convulsive” Nonepileptic Seizures Have a Characteristic Pattern of Rhythmic Artifact Distinguishing Them from Convulsive Epileptic Seizures. Epilepsia 2004; 45(11):1344-1350.
2. Hjorth B. EEG analysis based on time domain properties. Electroencephalography and Clinical Neurophysiology 1970; 29, 306-310.
3. Clinical Applications of Computer Analysis of EEG and other Neurophysiological Signals, EEG Handbook revised series, Vol 2, FH Lopes da Silva, WS van Leeuwen and A Remond (Eds), Elsevier Science, Amsterdam, 1987.
4. Baharav A, Shinar Z, and Akselrod S. Muscle Activity During Sleep: A Quantitative Electromyography Analysis during Standard Polysomnography. SLEEP 2003; 26 (Absts):A393.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A computer implemented method of distinguishing epileptic (ES) from non-epileptic pseudoseizures (PS) derived from motor activity of the limbs of a subject.

2. The method of claim 1, comprising:
measuring and accumulating an electronic data stream representing said motor activity.

3. The method of claim 2, further comprising:
quantifying differences within said electronic data stream to distinguish said epileptic (ES) from non-epileptic pseudoseizures (PS).

4. The method of claim 3, wherein said measuring is provided by a sensor device.

5. The method of claim 4, wherein said said sensor device comprises at least one of the following: accelerometer, electromyographic electrodes or optoelectronic distortion sensors.

6. The method of claim 4, further comprising:
placing said sensor device on a limb of said subject.

7. A system for diagnosing convulsions of a subject, said system comprising:
a sensor device for measuring and accumulating an electronic data stream representing motor activity; and
a processor for processing said electronic data to distinguish epileptic (ES) from non-epileptic pseudoseizures (PS).

8. The system of claim 7, wherein said sensor device is provided by at least one of the following types of devices: accelerometer, electromyographic electrodes or optoelectronic distortion sensors.

9. The system of claim 7, wherein said processing comprises:
approximate entropy (ApEn) technique or peak-to-peak amplitude of repeated patterns (PPARP) technique, or any combination thereof.

10. A computer program product comprising a computer useable medium having computer program logic for enabling at least one processor in a computer system to diagnose convulsions of a subject, said computer program logic comprising:
receiving electronic data representing motor activity; and
processing said electronic data to distinguish epileptic (ES) from non-epileptic pseudoseizures (PS).

11. The computer program product of claim 10, wherein said processing comprises:
approximate entropy (ApEn) technique or peak-to-peak amplitude of repeated patterns (PPARP) technique, or any combination thereof.
